(19) United States
(12) Patent Application Publication
(10) Pub. No.: US 2019/0030288 A1
(43) Pub. Date: Jan. 31, 2019

(54) MEDICAL ARTICLE SECUREMENT SYSTEM COMPRISING A FLAP AND A VERTICAL WALL HAVING MATING SURFACES OF A MECHANICAL FASTENER

(71) Applicant: 3M INNOVATIVE PROPERTIES COMPANY, St. Paul, MN (US)

(72) Inventors: Naimul Karim, Maplewood, MN (US); Jennifer N. Hanson, Woodbury, MN (US); Shilpi K. Sanghi, Stanford, CA (US); Jia Hu, Mounds View, MN (US)

(73) Assignee: 3M INNOVATIVE PROPERTIES COMPANY, St. Paul, MN (US)

(21) Appl. No.: 15/755,891

(22) PCT Filed: Oct. 31, 2016
(86) PCT No.: PCT/US16/59635

§ 371 Date: Feb. 27, 2018

Related U.S. Application Data
(60) Provisional application No. 62/249,542, filed on Nov. 2, 2015.

(51) Int. Cl. A61M 25/02

(52) U.S. Cl. CPC A61M 25/02; A61M 2025/0266; A61M 2025/026

(57) ABSTRACT

A medical article securement system for securing a medical article. The system can include a patch; a vertical wall comprising a first major surface positioned to face the medical article and a second major surface comprising a first mating surface of a mechanical fastener; and a flap comprising a first major surface positioned to face the medical article, at least a portion of the first major surface comprising a second mating surface of a mechanical fastener configured to engage the first mating surface of the vertical wall.

### FIELD

The present disclosure generally relates to medical article securement systems comprising a flap and a vertical wall having mating surfaces of a mechanical fastener for securing a medical article to the body of a patient, and particularly, for securing medical tubing, such as various catheter systems or other elongated devices to the body of a patient.

### BACKGROUND

Various medical treatments often require the use of medical articles and tubing. In many cases the devices or tubing must be secured to a patient's body. For example, it can be necessary to introduce fluids and liquid medications directly into a blood vessel of a patient. For short term general use, a simple intravenous (IV) catheter can be placed onto a patient's arm. For longer term and more specialized needs, central line catheters or other devices are used. In another example, a urinary catheter (such as a Foley catheter) may be necessary for draining urine from a patient's bladder.

Healthcare providers often secure catheters, other devices or tubing to patients during hospital stays or in-home care. Securing the devices aids in proper positioning, which inhibits dislodgement or tangling that may cause leakage or interruptions in medication dosing. Securement of such devices also minimizes patient discomfort and reduces the risk of infection.

In order to keep a catheter or other medical articles or tubing properly positioned for the duration of treatment, the medical article may be secured to the patient in a variety of ways. One common way of securing a medical article or tubing is by taping the catheter or medical line to the patient's skin. However, taping can be time consuming and labor intensive. Tape can also collect contaminants and must be frequently removed and replaced. In addition, taping is not necessarily effective in securing a medical article or catheter in place, and removal of the tape may cause undesired motion of the device or catheter. Sutures have also been used to attach a catheter to a patient. With sutures, the catheter is stitched onto the skin. Sutures, however, can be a source of infection, can cause pain and inflammation, and can make it more difficult to clean around the incision site. Sutures also require time and skill to place, and can cause scarring.

Various other securement devices have been developed to obviate some of the fallbacks associated with the use of tape and sutures. Some existing securement devices are generally designed for a specific type or size of catheter or medical article. As a result, multiple securement devices may be needed to accommodate different types or sizes of catheters, e.g., in hospitals and clinical settings. This can add to the cost and complexity of sourcing, inventory, storage, and selection of the securement devices. Additionally, many securement devices still suffer effects of patient movement in which tubing may become kinked or pinched and restrict flow of medication, blood, or urine.

There remains a need for securement devices that accommodate varying sizes of medical articles or tubing and allow patient movement while not disrupting or kinking the tubing.

### SUMMARY

The present disclosure is generally directed to medical article securement devices, systems, and methods, and particularly, to universal securement devices, systems, and methods that are adapted to accommodate and reliably secure a large variety of shapes and sizes of catheter systems or other medical articles, particularly elongated medical articles such as medical tubing. The securement devices, systems, and methods of the present disclosure are generally robust, easy to use, and are designed to facilitate coupling and decoupling a medical article to and from the system, while also providing means for reliably retaining a medical article, such as medical tubing (e.g., of a catheter system), for a desired treatment period.

Some aspects of the present disclosure provide a medical article securement system for securing a medical article. The system can include a patch comprising a first major surface, and a second major surface, opposite the first major surface, comprising a skin-contact adhesive. The system can further include a vertical wall, the vertical wall comprising a first major surface positioned to face the medical article and a second major surface opposite the first major surface, the second major surface comprising a first mating surface of a mechanical fastener. The system can further include a flap comprising a fixed end coupled to the patch, and a free end that is movable with respect to the patch between a first position in which the free end is not positioned in overlapping relationship with the vertical wall and a second position in which the free end is positioned in overlapping relationship with the vertical wall to couple at least a portion of the medical article to the patch. At least the free end of the flap can include a first major surface positioned to face the medical article, at least a portion of the first major surface comprising a second mating surface of a mechanical fastener configured to engage the first mating surface of the vertical wall, and a second major surface, opposite the first surface, positioned to face away from the medical article.

Other features and aspects of the present disclosure will become apparent by consideration of the detailed description and accompanying drawings.

### BRIEF DESCRIPTION OF THE DRAWINGS

FIG. 1 is a front perspective view of a medical article securement system according to one embodiment of the present disclosure.

FIG. 2 is a side perspective view of the medical article securement system of FIG. 1.

FIG. 3 is a side perspective view of a medical article securement system of FIGS. 1 and 2, shown with a medical article secured.

FIG. 3B is a schematic side cross-sectional view of the medical article securement system of FIG. 3A, with the medical article shown secured.

FIG. 4 is a top plan view of a medical article securement system according to another embodiment of the present disclosure.

FIG. 5 is a top plan view of the medical article securement system of FIG. 4, shown with a medical article unsecured.

FIG. 6 is a top plan view of the medical article securement system of FIGS. 4 and 5, shown with the medical article secured.

FIG. 7 is a perspective view of a medical article securement system according to another embodiment of the present disclosure.

### DETAILED DESCRIPTION

The present disclosure generally relates to medical article securement devices and systems and methods for safely and reliably securing a medical article, such as medical tubing, upon a desired location of a patient's body. The medical article securement systems can be universal to accommodate and reliably secure a large variety of medical articles or class of medical articles (e.g., Foley catheters and peripherally inserted central catheters (PICCs)), and can be particularly useful for securing medical articles that need to be secured to a patient over a prolonged period of time, such as weeks or months.

Examples of medical articles that can be employed with the medical article securement devices and systems of the present disclosure include, but are not limited to, medical tubing or fluid supply lines, other similar articles, or combinations thereof. Examples of medical tubing can include, but are not limited to, catheters (e.g., urinary catheters (e.g., Foley catheters), intravenous (IV) catheters, central venous catheters (CVCs), peripherally inserted central catheters (PICCs), arterial catheters, chest tubes, drainage tubes, infant umbilical catheters, and dialysis catheters.

### Definitions

The term “a”, “an”, and “the” are used interchangeably with “at least one” to mean one or more of the elements being described.

The term “and/or” means either or both. For example “A and/or B” means only A, only B, or both A and B.

The terms “including,” “comprising,” or “having,” and variations thereof, are meant to encompass the items listed thereafter and equivalents thereof as well as additional items.

Unless specified or limited otherwise, the term “coupled” and variations thereof are used broadly and encompass both direct and indirect couplings.

Relative positional terms, such as “top,” “bottom,” “upper,” and the like, are only used to describe elements as they relate to one another, but are in no way meant to indicate or imply necessary or required orientations of the apparatus, or to specify how the invention described herein must be used, mounted, displayed, or positioned in use.

The terms “longitudinal” and “axial” are used to refer to a direction or axis that is generally parallel to the direction in which the medical article extends and generally parallel to the overall direction of fluid flow, e.g., along a catheter line.

The term “lateral” is used to refer to a direction or axis that is perpendicular to the longitudinal axis or direction and is used to represent side-to-side motion of a medical article.

The terms “vertical” and “normal” are used to refer to a direction or axis that is normal to both the longitudinal and lateral directions or axes, as well as to the surface of a patient's skin when the medical article securement system is coupled to the patient's skin, and is used to represent the direction of motion toward and away from the skin surface.

The phrase “vertical wall” is used to refer to a structure having a vertical surface that extends in or has dimensions in the vertical direction. For example, such a vertical wall can have a vertical surface that extends in and has dimensions in the vertical and lateral directions, in the vertical and longitudinal directions, or in the vertical direction and a combination of the lateral and longitudinal directions. That is, in some embodiments, the vertical wall can be oriented substantially parallel with the lateral direction, can be oriented substantially parallel with the longitudinal direction, or can be oriented at an angle (e.g., an acute angle) with respect to both the lateral direction and the longitudinal direction.

The term “proximal” and “distal” are used to represent relative axial directions, relative to a medical practitioner operating or holding the medical article. That is, the term “distal” is used to refer to the direction away from the medical practitioner (and toward an insertion site on the patient and inside the patient's body), and the term “proximal” is used to refer to the direction toward the medical practitioner (and toward the outside of the patient's body, away from the insertion site). For example, the distal end of a catheter is inserted into the patient, while the proximal end extends exterior of the patient toward the medical practitioner. The distal end of the medical article securement system refers to the end of the system that is configured to be oriented toward the distal end of the medical article to which it will be coupled, and the proximal end of the medical article securement system refers to the end of the system that is configured to be oriented toward the proximal end of the medical article.

The terms “layer,” “sheet,” and “dressing,” or variations thereof, are used to describe an article having a thickness that is small relative to its length and width.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The term “repositionable” refers to the ability of an article or surface to be, at least initially, repeatedly coupled to (e.g., adhered to) and removed from a surface or substrate without substantial loss of coupling capability (e.g., adhesion) and without damage to either surface (e.g., article or underlying substrate) being coupled together. By way of example, some pressure-sensitive adhesives and mechanical fasteners are repositionable.

The phrase “mechanical fastener” or “touch fastener” generally refers to a fastener that includes two mating, or engagement, surfaces configured to be applied to one another, each mating surface having a plurality of engagement structures or features, such that engagement structures on one mating surface are configured to engage with the engagement structures on the opposing mating surface. In some embodiments, the mechanical fastener can include two flexible mating strips or layers. In some embodiments, the mechanical fastener can include a first mating surface comprising tiny, stiff protrusions shaped like hooks that are configured to engage a second mating surface comprising pliable loops (i.e., a “hook and loop fastener,” or “hook and pile fastener”). In some embodiments, the mechanical fastener can include inter-engaging hooks (e.g., self-engaging hooks) on both mating surfaces (i.e., a “hook and hook fastener” or a “self-engaging hook fastener”).

“Peel force” refers to the force needed to “peel” one surface from another surface at an angle with respect to the plane between the surfaces. Adhesive peel force can be measured using the ASTM method referenced in the “Adhesives” section below. Peel force between mating surfaces of a mechanical fastener can be measured using ASTM D5170-98 (2015)—Standard Test Method for Peel Strength (“T” Method) of Hook and Loop Touch Fasteners.

“Shear strength” (or “shear force”) refers to the resistance to forces that cause, or tend to cause, two contiguous parts of a body to slide relatively to each other in a direction parallel to their plane of contact. That is, shear strength is the amount of force required to move one surface relative to another surface when the two surfaces are pulled in opposite directions parallel to their plane of contact. Adhesive shear force can be measured using the ASTM method referenced in the “Adhesives” section below. Shear force between mating surfaces of a mechanical fastener can be measured using ASTM D5169-98(2015)—Standard Test Method for Shear Strength (Dynamic Method) of Hook and Loop Touch Fasteners.

The term “flexible” can generally be used to refer to a material that is drapable. That is, a section of material 5 cm×15 cm when held upright (long end up) folds over under its own weight to drop the opposite end to or below the holder, when performed at ambient conditions. The term “rigid” can generally be used to refer to a material that is essentially non-drapable. That is, a section of material 5 cm×15 cm when held upright (long end up) stands straight up with little or no deflection, when performed at ambient conditions. In some embodiments, rigid materials can show less than 20 degrees of deflection from vertical. “Semi-rigid” materials can be those that exhibit more than 20 degrees of deflection but whose opposite end does not drop below the holder.

The medical article securement systems of the present disclosure include one or more flaps (i.e., flexible flaps) and one or more vertical walls, wherein a flap and a corresponding vertical wall have complementary mating surfaces of a mechanical fastener, such that the flaps can be repositionable as desired, while providing a reliable securement system. The vertical wall can be flexible too and, in some embodiments, can be formed of the same or a similar material as the flap, as long as the vertical wall has a vertical surface that is oriented to face away from the medical article and to be coupled to a free end of the flap after the free end of the flap has been wrapped about at least a portion of the medical article.

When the medical article is positioned adjacent the vertical wall, particularly, such that a free end of the flap is on an opposite side of the medical article from the vertical wall, and the flap is wrapped about at least a portion of the medical article (e.g., about at least a portion of a circumference of a medical tubing), the mating surface on the flap engages with its complementary mating surface on the vertical wall. As a result, when a secured medical article is subjected to a displacement force (e.g., in a vertical or normal direction), the shear component (i.e., between complementary surfaces of the mechanical fastener, i.e., between adjacent surfaces of the flap) is maximized, while the peel component is minimized. Mechanical fasteners are more resistant to shear forces than peel forces and consequently have higher shear strength than peel strength. As a result, the systems of the present disclosure employ mechanical fasteners on flaps and vertical walls in such a way that the shear strength of the mechanical fastener is exploited.

On the contrary, in some existing systems, a flap may include a mating surface of a mechanical fastener on its underside that can be folded over an article and engaged with a complementary mating surface on a flat surface on which the article resides. In such a configuration, when a secured article is subjected to a displacement force (e.g., in a vertical or normal direction), the peel component (i.e., between complementary surfaces of the mechanical fastener) is maximized, and the overall securement of the article would be weaker for the same mechanical fastener, because the shear strength of the mechanical fastener would not be exploited.

In some embodiments, medical article securement systems of the present disclosure can provide a variety of unique advantages and benefits. For example, by employing mechanical fasteners, the systems are able to be opened and closed multiple times (i.e., the securement means are repositionable), while remaining on the patient, which is typically not possible with adhesive tape-based systems. In addition, the fastening means of mechanical fasteners are not compromised if soiled or contaminated (e.g., as compared to adhesive tape-based systems). Furthermore, the systems of the present disclosure are universal in that the systems can accommodate and secure a wide range of sizes and shapes of medical articles.

Furthermore, because at least one flap of the systems of the present disclosure includes a mating surface of a mechanical fastener that is complementary to and engages a mating surface on the vertical wall, at least a portion of a medical article can be secured in a way that exploits the shear strength of the mechanical fastener, such that the system has a higher resistance to displacement forces on the medical article (e.g., in a vertical or normal direction).

In addition, because the flap and the vertical wall of systems of the present disclosure include complementary mating surfaces of a mechanical fastener, no matter how the flap is positioned around the medical article to secure it, enhanced securement will be provided (i.e., the shear strength of the mechanical fastener will be exploited) as long as the mating surface of the mechanical fastener on the flap contacts the complementary mating surface of the mechanical fastener on the vertical wall, thereby making application of the systems of the present disclosure more fool-proof, consistent, and user-independent.

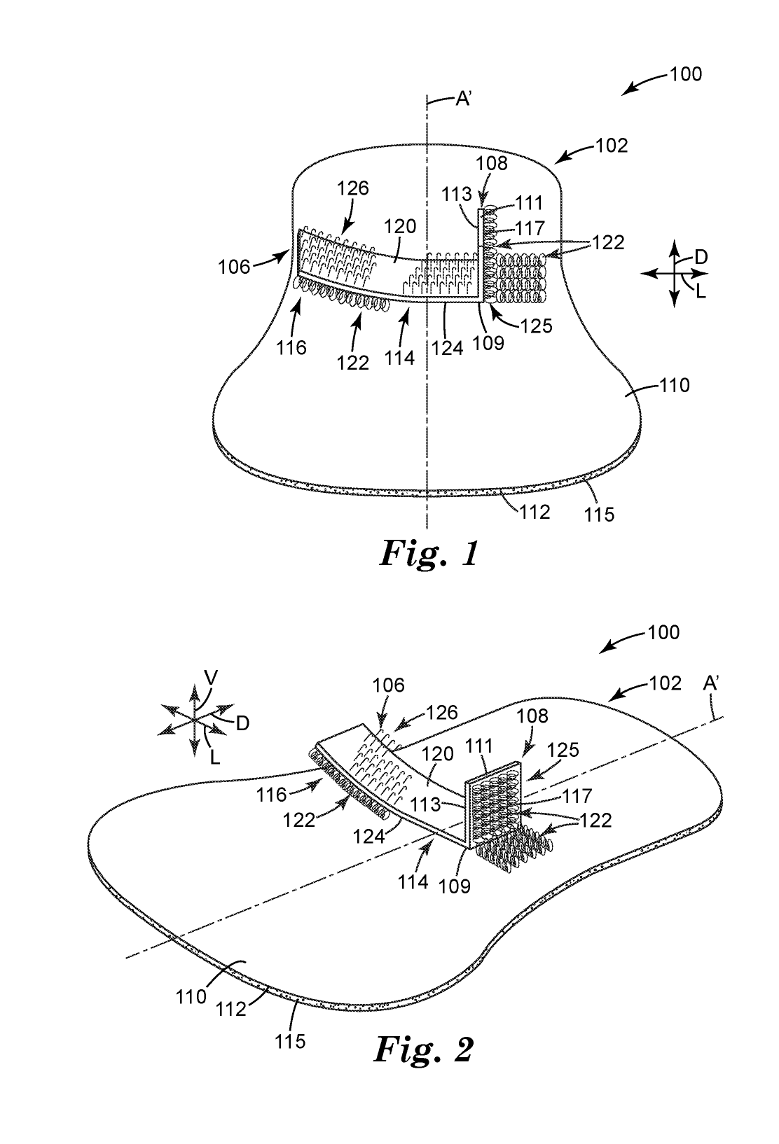

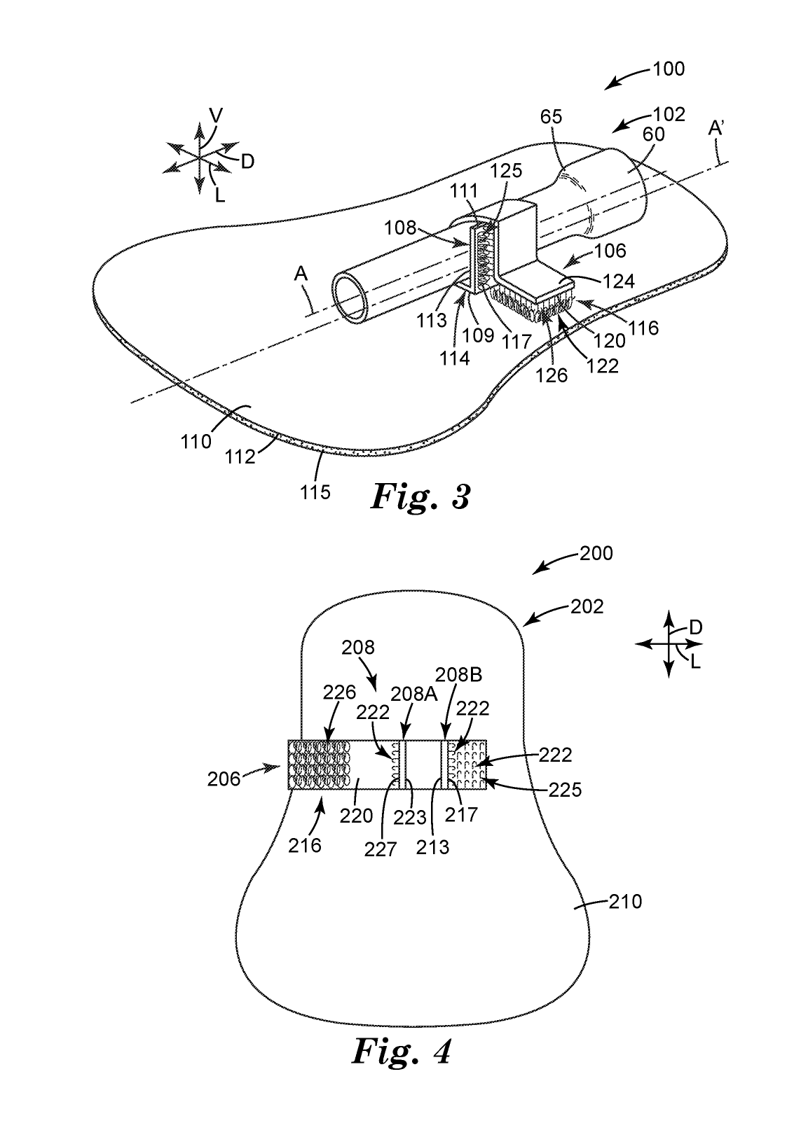

FIGS. 1-3 illustrate a medical article securement system 100 according to one embodiment of the present disclosure. The medical article securement system 100 is shown in FIG. 3 as securing a medical article 60, which is shown by way of example as being medical tubing, such as a catheter. The medical article 60 has a longitudinal axis A (see FIG. 3) that defines a longitudinal direction D (see FIGS. 1-3). The system 100 has a longitudinal axis A′ that is configured to be substantially aligned with the longitudinal axis A of the medical article 60 when the medical article 60 is positioned on or coupled to the system 100.

As shown in FIGS. 1-3, the system 100 can include a patch (or base dressing, or base layer) 102 configured to receive the medical article 60 such that the longitudinal axis A of the medical article 60 is substantially aligned with the longitudinal axis A′ of the system 100. The system 100 can further include one or more flaps (e.g., lateral flaps) 106 and one or more vertical walls 108. The system 100 of FIGS. 1-3 is shown as including only one flap 106 and one vertical wall 108 opposite the flap 106; however, other embodiments of systems of the present disclosure can include multiple flaps and/or vertical walls. When describing the system 100 of FIGS. 1-3, one flap 106 and one vertical wall 108 will be described for simplicity, but it should be understood that the description can apply to as many flaps that are employed in a given system.

The flap 106 and the vertical wall 108 can be coupled to the patch 102 and/or integrally formed with the patch 102, and the patch 102 can be adhered to skin. The flap 106 and the vertical wall 108 can be used to secure the medical article 60 to the system 100 and the patient's skin. As shown in FIGS. 1-3, in some embodiments, the flap 106 can be positioned with respect to the patch 102 and oriented with respect to the longitudinal direction D, such that the flap 106 can secure the medical article 60 in such a way that it inhibits movement of the medical article 60 in at least a vertical direction V, but also potentially in a lateral direction L. In addition, in some embodiments, the vertical wall 108 can be oriented substantially parallel with the longitudinal direction D, such that its vertical surfaces are laterally-facing; however, the vertical wall 108 need not be oriented longitudinally.

In some embodiments, the flap 106 and/or the vertical wall 108 can be formed of the same backing material as one another. In some embodiments, one or both of the flap 106 and the vertical wall 108 can be formed of the same backing material as the patch 102. In some embodiments, the flap 106 and the vertical wall 108 may be formed of the same backing material but may be formed of a different backing material than the patch 102. Various additional details regarding backings of the present disclosure are described in greater detail below under the section entitled, “Backings.”

As shown in FIGS. 1-3, the patch 102 includes a first major surface 110 configured to face away from the patient's skin, and a second major surface 112 opposite the first major surface 110 that comprises a skin-contact adhesive 115 for adhering to the skin. Although the patch 102 is illustrated as having rounded edges and one larger end, it should be understood that the patch 102 can take on a variety of shapes and sizes, depending on the shapes and configurations of the other elements of the system 100 and the medical article 60 to be coupled to the system 100. In some embodiments, the patch 102 comprises a laminated structure comprising one or more of a fabric, a woven fibrous web, a nonwoven fibrous web, a knit, a polymeric film, or combinations thereof

The skin-contact adhesive 115 is generally a pressure-sensitive adhesive, and particularly is a pressure-sensitive adhesive that is capable of securely but releasably adhering or bonding to skin (e.g., mammalian skin). The skin-contact adhesive 115 is also generally safe and non-toxic. Skin-contact adhesive layers will generally be selected according to the desired end use of the patch 102. In some embodiments, the patch 102 can include more than one skin-contact adhesive 115. Where the patch 102 comprises more than one skin-contact adhesive layer 115, each skin-contact adhesive layer 115 may be selected independently of each other with regard to material and thickness used. Examples of suitable adhesives include acrylates, silicones, polyisobutylenes, synthetic rubber, natural rubber, and copolymers and mixtures thereof Acrylates and silicones can be preferred skin-contact adhesives 115. In general, the skin-contact adhesive 115 should cause little or no irritation or sensitization of the skin during the intended wear period. Examples of skin-contact adhesives 115 that can be employed with the systems of the present disclosure include, but are not limited to, the adhesives described in U.S. Pat. Nos. RE24,906; 3,389,827; 6,103,369 and 4,499,896, which are incorporated herein by reference. In addition, silicone adhesives such as those described in U.S. Patent Publication No. 2011/0212325, which is incorporated herein by reference, can also be employed.

In some embodiments, e.g., in embodiments employing silicone adhesives, the patch 102 and the skin-contact adhesive 115 can be perforated to provide openings from the first major surface 110 of the patch 102 all the way through the second major surface 112 and the skin-contact adhesive 115, which can enhance permeability of the patch 102 and can minimize moisture build-up at the skin surface underlying the patch 102.

In some embodiments, the system 100 can further include one or more release liners that can provide a release layer or surface to the skin-contact adhesive 115 on the second major surface 112 of the patch 102 prior to use. Examples of liners suitable for use with systems of the present disclosure can include, but are not limited to, kraft papers, polyethylene, polypropylene, polyester, or combinations thereof. Such liners can be coated with release agents, such as fluorochemicals, silicones, or other suitable low surface energy materials. Other adhesives and release liner combinations known to those of ordinary skill in the art can be employed in the systems of the present disclosure.

The flap 106 and/or the vertical wall 108 can be coupled to the patch 102 using a variety of coupling means including, but not limited to, one or more of adhesives, cohesives, welding (e.g., sonic [e.g., ultrasonic] welding), any thermal bonding or heat sealing technique (e.g., heat and/or pressure applied to one or both of the components to be coupled), other suitable coupling means, or combinations thereof. Additionally or alternatively, in some embodiments, at least a portion of the flap 106 and/or the vertical wall 108 can be integrally formed with the patch 102.

The flap 106 can be flexible so as to easily accommodate and secure the medical article 60, or any medical article of interest, and so as to be able to be wrapped about at least a portion of the medical article 60. For example, in some embodiments, the medical article 60 can include medical tubing, and the flap 106 can be wrapped about at least a portion of a circumference of the medical tubing.

As shown, the flap 106 can include a fixed end 114 coupled to the patch 102, and a free end 116 that is movable with respect to the patch 102 and configured to be wrapped around (i.e., has a sufficient length to be wrapped around) at least a portion of the medical article 60, and in some embodiments, configured to overlap, or cross, the longitudinal axis A′ of the system 100 (and the longitudinal axis A of the medical article 60) to secure at least a portion of the medical article 60 to the patch 102 (i.e., to the first major surface 110 of the patch 102).

The vertical wall 108 can include a base 109 located adjacent or toward, or coupled to, the first major surface 110 of the patch 102, and a top 111 spaced a vertical distance away from the first major surface 110 of the patch 102, such that the vertical wall 108 has a height in the vertical direction and stands in a vertical orientation with respect to the patch 102. That is, the vertical wall 108 can extend, or be oriented, substantially orthogonally with respect to the patch 102 (e.g., the first major surface 110 thereof), such that the vertical wall 108 stands up from the first major surface 110 of the patch 102. In some embodiments, the height of the vertical wall 108 (i.e., from its base 109 to its top 111) can be less than half of the length of the flap 106 (i.e., in a direction extending between the fixed end 114 and the free end 106 of the flap 106); and in some embodiments, less than one third of the length of the flap 106.

As shown, the vertical wall 108 can further include a first major surface (i.e., a first vertical surface) 113 positioned to face the medical article 60 (i.e., when the medical article 60 is coupled to the system 100), such that the medical article can be positioned adjacent a first side of the vertical wall 108. The vertical wall 108 can further include a second major surface (i.e., a second vertical surface) 117 opposite the first major surface 113, and the second major surface 117 can include a first mating surface 122 of a mechanical fastener 125.

The vertical wall 108 need not be rigid and can even be flexible, as mentioned above, but is configured (i.e., by material makeup, dimensions, thickness, etc. to have sufficient stiffness) to stand up away from the first major surface 110 of the patch 102 when desired, i.e., when the medical article 60 is to be secured and positioned up against the first major surface 113 of the vertical wall 108.

The flap 106 can be movable with respect to the patch 102 (and/or the medical article 60) between a first position in which the free end 116 is not positioned in overlapping relationship with the vertical wall 108 (see FIGS. 1 and 2) and a second position in which the free end 116 is positioned in overlapping relationship with the vertical wall 108 (see FIG. 3).

The flap 106, and particularly, at least the free end 116 of the flap 106, can include a first major surface 120 configured to face the medical article 60 (i.e., at least when the free end 116 of the flap 106 is in the second position); and a second major surface 124, opposite the first major surface 120, configured to face away from the medical article 60 (i.e., at least when the free end 116 of the flap 106 is in the second position). At least a portion of the first major surface 120 can include a second mating surface 126 of the mechanical fastener 125 configured to engage the first mating surface 122 of the vertical wall 108 (i.e., when the flap 106 is in its second position). The vertical wall 108, and particularly, the base 109 thereof, is located relative to the fixed end 114 of the flap 106, such that the second major surface 117 of the vertical wall 108 comprising the first mating surface 122 faces away from the flap 106. As a result, when the flap 106 is in its first position, at least the free end 116 of the flap 106 extends in a direction outwardly away from the first major surface 113 of the vertical wall 108. Furthermore, the vertical wall 108 and at least the free end 116 of the flap 106 can be positioned to receive at least a portion of the medical article 60 therebetween.

In some embodiments, as shown, the fixed end 114 of the flap 106 can have sufficient length along the first major surface 110 of the patch 102, and the medical article 60 can be positioned atop the fixed end 114 of the flap 106. However, this need not be the case, and in some embodiments, the medical article 60 can simply be positioned between the fixed end 114 of the flap 106 and the vertical wall 108 (i.e., the base 109 thereof).

The relative positioning of the flap 106 and the vertical wall 108, along with the arrangement of the complementary first and second mating surfaces 122 and 126 of the mechanical fastener 125 allows securement of the medical article 60 in a way that exploits the shear strength of the mechanical fastener 125. When the free end 106 of the flap 106 is positioned over the vertical wall 108 (and the medical article 60), the second mating surface 126 on the first major surface 120 of the flap 106 engages the first mating surface 122 on the second major surface 117 of the vertical wall 108. The second major surface 117 of the vertical wall 108 is a vertical surface, and the free end 116 of the flap 106 overlaps and engages the vertical second major surface 117 of the vertical wall 108 when in its second position. As a result, when the medical article 60 is subjected to a displacement force, and particularly, a displacement force in the vertical direction V, the mechanical fastener 125, in the region where the free end 116 of the flap 106 is engaged with the vertical wall 108, will be subjected primarily to shear forces, thereby exploiting the relatively greater strength of the mechanical fastener in shear mode, as opposed to in peel mode, resulting in very reliable securement of the medical article 60.

As mentioned above and as shown in FIGS. 1-3, in some embodiments, the flap 106 and the vertical wall 108 can be integrally formed, such that the flap 106 and the vertical wall 108 are provided by the same piece of material, and such that the vertical wall 108 is formed by a first end of the flap 106, and the free end 116 of the flap 106 is formed by a second end of the flap 106 that is opposite the first end (and the vertical wall 108). In such embodiments, the first major surface 120 of the flap 106 is the same surface that forms the first major surface 113 of the vertical wall 108, and the second major surface 124 of the flap 106 is the same surface that forms the second major surface 117 of the vertical wall 108. As a result, in some embodiments, the second major surface 124 of the flap 106 can also include the first mating surface 122. In addition, the first major surface 113 of the vertical wall 108 can also include the second mating surface 126. In such embodiments, a middle portion of the material can be coupled to the patch 102 (or integrally formed with the patch 102), and this middle portion can form the fixed end 114 of the flap 106. In addition, the material can be coupled to the patch 102 in such a way that the vertical wall 108 is formed on one side of the fixed end 114 of the flap 106, and the free end 116 of the flap 106 is formed on an opposite side of the fixed end 114 of the flap 106. As a result, the first major surface 120 of the free end 116 of the flap 106 is positioned to face the medical article 60 and the vertical wall 108, and the second major surface 117 of the vertical wall 108 faces away from the medical article 60 and the flap 106.

By way of example only, the first mating surface 122 is schematically illustrated in FIGS. 1-3 as including piles or loops, and the second mating surface 126 is schematically illustrated as including hooks or pegs. However, it should be understood that any appropriate mechanical fastener engagement surface or mechanism can be employed, as long as the first mating surface 122 and the second mating surface 126 are complementary mating surfaces of a mechanical fastener that are configured to engage one another.

As shown in FIGS. 1-3, in some embodiments, the flap 106 (or flaps, if more than one is employed) can extend laterally in a lateral direction L, and the vertical wall 108 can be oriented substantially longitudinally, such that the first major surface 113 and the second major surface 117 have dimensions in the longitudinal and vertical directions. In some embodiments, the flap 106 can be elongated in the lateral direction L, but this need not be the case. That is, a flap 106 can extend laterally as long as the flap 106 is oriented to include a dimension in the lateral direction L. As a result, the flap 106 can be positioned to wrap laterally about the vertical wall 108, and the medical article 60, i.e., with respect to the longitudinal axis A of the medical article 60.

As shown in FIG. 1, in some embodiments, the flap 106 can include a length that is greater than its width and its thickness, and the length of the flap 106 can be oriented, or extend, at least partially laterally with respect to the longitudinal direction A′. In addition, in some embodiments, the vertical wall 108 can include a length that is greater than its width and its thickness, and the length of the vertical wall 108 can be oriented, or extend, substantially longitudinally, i.e., substantially parallel to the longitudinal axis A′.

In such embodiments, the flap 106 can be movable with respect to the vertical wall 108 (and/or the medical article 60) between its first position in which the free end 116 does not overlap the longitudinal axis A′ of the system 100 (or the longitudinal axis A of the medical article 60) and a second position in which the free end 116 is positioned to overlap the longitudinal axis A′ of the system (or the longitudinal axis A of the medical article 60). Said another way, in some embodiments, the flap 106 can be movable with respect to the vertical wall 108 (and/or the medical article 60) between a first position in which the free end 116 does not cross the longitudinal axis A′ of the system 100 (or the longitudinal axis A of the medical article 60) and a second position in which the free end 116 is positioned to laterally cross the longitudinal axis A′ of the system (or the longitudinal axis A of the medical article 60).

In some embodiments, the system 100 can be configured to secure the medical article 60 in the region of an irregular feature to enhance securement of the medical article 60. That is, in some embodiments, the system 100 can be configured (e.g., the flap 106 can be shaped, sized and/or positioned) to facilitate coupling an irregular feature of the medical article 60, for example, by being coupled to a portion of the medical article 60 that comprises the irregular feature or by being coupled to a portion of the medical article 60 adjacent the irregular feature. For example, as shown in FIG. 3, the medical article 60 includes medical tubing that includes a change in diameter 65. As a result, it can be useful to secure a portion of the medical article 60 comprising the irregular feature, e.g., by coupling the medical article 60 to the system 100, such that the flap 106 and the vertical wall 108 are located adjacent the change in diameter 65. Such positioning of the flap 106 and the vertical wall 108 relative the medical article 60 can further secure the medical article 60 by inhibiting movement of the medical article 60 not only in the vertical direction V and the lateral direction L, but also in the longitudinal direction D, or at least in one of a proximal or a distal longitudinal direction.

Medical articles can include other irregular features over or adjacent which it can be useful to position the flap 106 and the vertical wall 108. For example, an irregular feature of a medical article can include, but is not limited to, a connection point for multiple lumens (e.g., bifurcation point, trifurcation point, etc.), a change in diameter (e.g., a step-change or multi-step change), a protrusion (e.g., a knob, a dial, a meter, a connector), a constriction, or any other feature where a medical article may deviate from a uniform or regular shape, such as a tube or cylinder having a substantially uniform diameter.

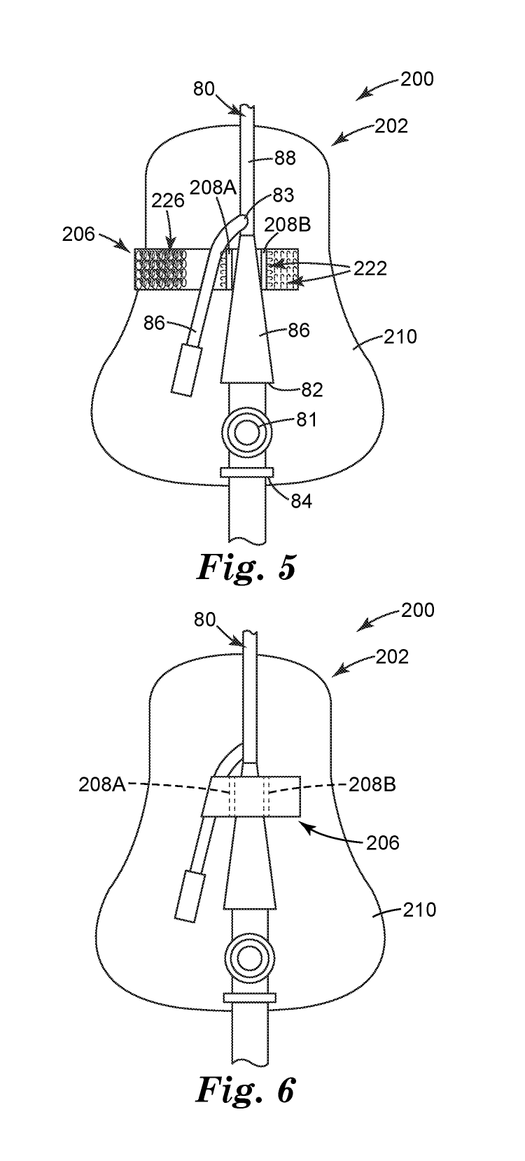

The change in diameter 65 of the medical article 60 of FIG. 3 is shown by way of example only. However, in some embodiments, the vertical wall 108 can be oriented on the patch 102 to provide a longitudinal stop for the medical article 60. For example, in some embodiments, the medical article 60 can include a multi-lumen joint (or hub, or connection point), and in such embodiments, the vertical wall 108 can be oriented on the patch 102 such that when the medical article 60 is coupled to the system 100, the vertical wall 108 provides a longitudinal stop against the multi-lumen joint to inhibit longitudinal movement of the medical article 60 with respect to the patch 102 (and the subject's skin), at least in one of a proximal and distal longitudinal direction. One example of this is illustrated in FIGS. 5 and 6, which are described in greater detail below.

As further shown in FIGS. 1-3, in some embodiments, the patch 102, e.g., the first major surface 110 of the patch 102, can include at least one of the first mating surface 122 and the second mating surface 126, at least in a region located adjacent the vertical wall 108. Particularly, the first major surface 110 of the patch 102 can include a mating surface 122, 126 of the mechanical fastener 125 in the region adjacent the second major surface 117 of the vertical wall 108, i.e., on an opposite side of the vertical wall 108 from the flap 106. For example, as shown in FIGS. 1-3, in some embodiments, at least a portion of the first major surface 110 of the patch 102 can include the first mating surface 122, such that after the flap 106 is positioned to overlap and engage with the vertical wall 108 (and is positioned over the medical article 60), the free end 116 of the flap 106 can be further secured to the first major surface 110 of the patch 102 by engaging the second mating surface 126 on the first major surface 120 of the flap 106 with the first mating surface 122 on the first major surface 110 of the patch 102. Alternatively, in some embodiments in which the second major surface 124 of the flap 106 includes the first mating surface 122, the patch 102 can include the second mating surface 126, such that the first mating surface 122 on the second major surface 124 of the flap 106 can engage the second mating surface 126 on the patch 102 (e.g., if the flap 106 is further wrapped and tucked under so the second major surface 124 faces the first major surface 110 of the patch 102). Various combinations of these options are also possible.

In embodiments in which the first major surface 110 of the patch 102 includes the first mating surface 122 and/or the second mating surface 126, the mating surface(s) can be provided directly on the first major surface 110 of the patch 102, and/or can be provided by another layer, sheet or material that is coupled to the first major surface 110 of the patch 102.

If securement of the medical article 60 were to be accomplished by engaging the second mating surface 126 on the first major surface 120 of the flap 106 only with the first mating surface 122 on the first major surface 110 of the patch 102, then any displacement or decoupling force exerted on the medical article 60 in the vertical direction V, for example, would initiate a decoupling (i.e., disengagement) of the mechanical fastener 125 predominantly in peel mode. However, in securement systems of the present disclosure, the medical article 60 is secured by engaging the complementary first and second mating surfaces 122 and 126 on the vertical wall 108 and the flap 106, respectively, as described above. As a result, a similar vertical displacement force exerted on the medical article 60 initiates a decoupling (i.e., disengagement) of the mechanical fastener 125 predominantly in shear mode, i.e., thereby exploiting the relatively greater strength of the mechanical fastener 125 in shear mode, as opposed to in peel mode. As a result, the securement of the medical article 60 can be enhanced, as compared to other securement systems.

By way of example only, the flap 106 of FIGS. 1-3 is shown as being substantially linear and straight. However, in some embodiments, the flap 106 can have a non-linear shape, or include a bulbous end to facilitate being wrapped about medical articles and/or to increase the surface area of the free end 116 of the flap 106 to enhance coupling the flap 106 to the vertical wall 108 and/or patch 102 (e.g., after being wrapped about a medical article).

In some embodiments, the flap 106 can include a hinge, e.g., a living hinge, about which the free end 116 can pivot to be movable toward and away from the vertical wall 108. Such a hinge, for example, can separate the flap 106 from the patch 102 in embodiments in which the flap 106 is integrally formed with the patch 102.

In some embodiments, the flexible flap 106 can provide conformability, ease of handling, ease of application, ease of packaging, universality for many different medical articles, low cost, etc. Furthermore, in some embodiments, the system 100 can be free of any rigid components (e.g., rigid securement devices) that are more rigid than the flap 106. Examples of rigid components, and particularly, rigid securement devices, can include, but are not limited to, one or more of brackets, retainers, clips, posts, clamps, hooks, other typical rigid devices or structures, or a combination thereof

### Backings

Suitable backings for patches, flaps and/or vertical walls of the present disclosure can include, but are not limited to, one or more of a fabric, a woven fibrous web, a nonwoven fibrous web, a knit, a polymeric film, other familiar dressing materials, or combinations thereof. In some embodiments, the backing materials can include polymeric elastic films (e.g., transparent or non-transparent), and can include, but are not limited to, films formed of elastomeric polyurethanes, co-polyesters, polyethylenes, or combinations thereof. The backing can be a high moisture vapor permeable film, i.e., a backing with a relatively high moisture vapor transmission rate (MVTR). U.S. Pat. No. 3,645,835 describes methods of making such films and methods for testing their permeability. The backing can be constituted of natural or synthetic sources of raw materials.

The backings of patches of the present disclosure advantageously should transmit moisture vapor at a rate equal to or greater than human skin. In some embodiments, the patch backing can be adhesive-coated. In such embodiments, the adhesive-coated backing can transmit moisture vapor at a rate of at least 300 g/m²/24 hrs/37° C./100-10% RH, and in some embodiments, at least 700 g/m²/₂4 hrs/37° C./100-10% RH. The patch backing is generally conformable to anatomical surfaces. As such, when the patch is applied to an anatomical surface, it conforms to the surface even when the surface is moved.

The backing can be a flexible material. For example, the backing can be a film, paper, woven, knit, foam, nonwoven material, or a combination thereof, or one or more layers of film, paper, woven, knit, foam, nonwoven, or a combination thereof. In some embodiments, it can be desirable that at least a portion of backing is formed of a transparent material to allow for viewing of underlying skin, a medical device, and/or a target site.

By way of example only, in some embodiments, the backing of a patch of the present disclosure can be formed of a film available under the trade designation TEGADERM® from 3M Company, St. Paul, Minn.

Additional exemplary embodiments of medical article securement systems of the present disclosure will now be described with respect to FIGS. 4-7. FIGS. 4-7 illustrate various medical article securement systems of the present disclosure, wherein like numerals represent like elements. The medical article securement systems of FIGS. 4-7 share many of the same elements, features, and functions as the medical article securement system 100 described above with respect to FIGS. 1-3. Reference is made to the description above accompanying FIGS. 1-3 for a more complete description of the features and elements (and alternatives to such features and elements) of the embodiments illustrated in FIGS. 4-7. Any of the features described above with respect to FIGS. 1-3 can be applied to the embodiments of FIGS. 4-7, and vice versa.

FIGS. 4-6 illustrate a medical article securement system 200 according to another embodiment of the present disclosure. The system includes a patch 202 with a skin-contact adhesive (not shown), a flap 206, and two vertical walls 208.

FIG. 4 shows the flap 206 in the first position, without a medical article; FIG. 5 shows the flap 206 in the first position, with a medical article 80 positioned on the system 200; and FIG. 6 shows the flap 206 in the second position, i.e., at least partially overlapping the vertical wall 108 and the medical article 80, such that the medical article 80 is secured.

The system 200 is substantially the same as the system 100 of FIGS. 1-3, except that the system 200 of FIGS. 4-6 includes two vertical walls 208—i.e., a first vertical 208A and a second vertical wall 208B. Each of the vertical walls 208A, 208B includes a first major surface 113, 223 positioned to face a medical article and a second major surface 117, 227, respectively, opposite the first major surface 223 and positioned to face away from a medical article, when a medical article is coupled to the system 200.

In the system 200, the flap 206 and the two vertical walls 208 can all be provided by the same backing material that is coupled to the patch 202. Furthermore, as shown in FIG. 4, in the system 200, a first mating surface 222 of a mechanical fastener 225 is provided on the patch 202 by the same material that forms the flap 206 and the two vertical walls 208. Alternatively, as described above with respect to the system 100 of FIGS. 1-3, one or more of the flap 206 and the vertical walls 208 can instead be integrally formed with the patch 202. In further alternative embodiments, the first mating surface 222 can be formed directly on a first major surface 210 of the patch 202.

As further shown in FIG. 4, the second major surface 217 of the second vertical wall 208 includes the first mating surface 222, and a first major surface 220 of the flap 206 (and particularly a free end 216 thereof) includes a second mating surface 226 of the mechanical fastener 225. As a result, the second vertical wall 208B of the system 200 is similar to the vertical wall 108 of the system 100 of FIGS. 1-3, and the above disclosure regarding the vertical wall 108 of FIGS. 1-3 can apply to the second vertical wall 208B. For example, the flap 206 extends outwardly from the first major surface 213 of the second vertical wall 208B, i.e., opposite the second major surface 217 of the second vertical wall 208B. By way of example, the system 200 additionally includes the first vertical wall 208A. As shown in FIG. 4, the flap 206 extends outwardly adjacent the second major surface 227 of the first vertical wall 208A, rather than the first major surface 223 of the first vertical wall 208A.

As shown in FIG. 4, in some embodiments, the second major surface 227 of the first vertical wall 208A can also include the first mating surface 222, such that the second mating surface 226 on the first major surface 220 of the flap 206 can also engage the first mating surface 222 of the first vertical wall 208 when the flap 206 is in its second position.

In some embodiments, as shown, at least a portion of the medical article 80 (or any medical article of interest) desired to be secured by the system 200 can be positioned between the first vertical wall 208A and the second vertical wall 208B for added securement. In some embodiments, systems of the present disclosure can include a plurality of vertical walls, and the plurality of vertical walls can be positioned relative to one another to receive at least a portion of the medical article between two adjacent vertical walls.

By way of example only, the first mating surface 222 is schematically illustrated in FIGS. 4-6 as including hooks or pegs, and the second mating surface 226 is schematically illustrated as including piles or loops. However, it should be understood that any appropriate mechanical fastener engagement surface or mechanism can be employed, as long as the first mating surface 222 and the second mating surface 226 are complementary mating surfaces of a mechanical fastener that are configured to engage one another.

The medical article 80 of FIGS. 5-6 is illustrated by way of example only as being a Foley catheter. The medical article 80 illustrates several examples of irregular features, including a protrusion (e.g., a radial protrusion) 81, a change in diameter 82 or 84, and a multi-lumen joint (e.g., a bifurcation) 83. The medical article 80 further includes at least two proximal tubings 86 (each comprising a lumen for fluid communication) connected to the multi-lumen joint 83, and at least one distal tubing 88 (comprising a lumen for fluid communication), e.g., such that the multi-lumen joint 83 includes at least a bifurcation, but could also include a trifurcation, etc. The designation of the tubings 86 as proximal and the tubing 88 as distal is by way of example only.

As shown in FIGS. 5 and 6, the flap 206 and/or one or more of the vertical walls 208 can be positioned near at least one irregular feature of the medical article 80, such as one or more of the multi-lumen joint 83, the change in diameter 82, the change in diameter 84, and the protrusion 81, such that longitudinal movement of the medical article 80, i.e., in a longitudinal direction D can be inhibited when the medical article 80 is secured by the system 200. By way of example only, the medical article 80 is shown as being positioned on the system 200, such that at least one vertical wall 208—i.e., the first vertical wall 208A—is positioned adjacent the multi-lumen joint 83, and particularly between two proximal tubings 86, to provide a longitudinal stop (i.e., a proximal stop) for the medical article 80. While the first vertical wall 208A is illustrated as providing the longitudinal stop for the multi-lumen joint 83, it should be understood that, in some embodiments, the second vertical wall 208B can be used as the longitudinal stop, and the particular arrangement of the medical article 80 on the system 200 can depend on the medical article employed.

By way of example only, the first vertical wall 208A and the second vertical wall 208B are oriented substantially parallel with respect to one another (e.g., in a longitudinal direction D) and substantially perpendicularly with respect to the direction (e.g., a lateral direction L) in which the flap 206 extends. However, in some embodiments, one or both of the walls 208 need not be perpendicular with respect to the flap 206, and the walls 208 need not be parallel to one another. Furthermore, the vertical walls 208 need not be oriented in the longitudinal direction D, and the flap 206 need not extend in the lateral direction L. Rather, the orientation of the vertical walls 208 and the flap 206 can be chosen to be particularly suitable for a medical article of interest.

In use, as shown in FIGS. 5 and 6, the medical article 80 can be positioned on the patch 202 (e.g., on the first major surface 210 thereof), and the flap 206 can be moved from its first position (see FIG. 5) to its second position (see FIG. 6) to engage the second mating surface 226 on the first major surface 220 of the flap 206 with the first mating surface 222 on at least one of the second major surface 227 of the first vertical wall 208A and the second major surface 217 on the second vertical wall 208B. In addition, the second mating surface 226 on the flap 206 can engage the first mating surface 222 on the patch 202.

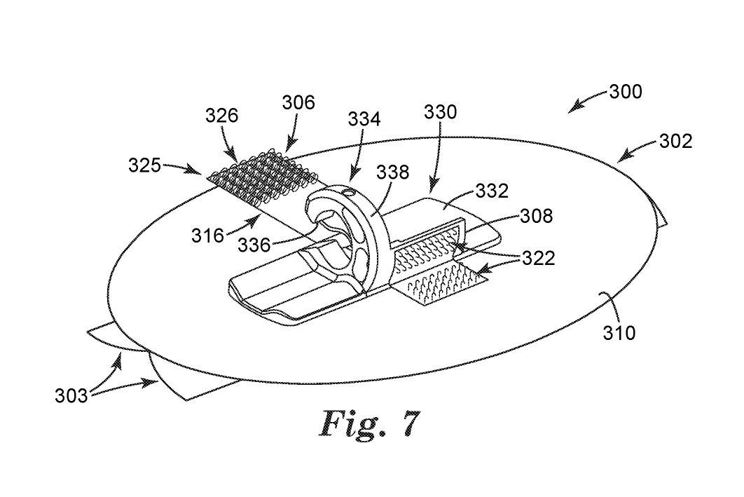

FIG. 7 illustrates a medical article securement system 300 according to another embodiment of the present disclosure. The system includes a patch 302 with a skin-contact adhesive (not shown), release liners 303, a flap 306, a vertical wall 308, and a rigid securement device 330. Two release liners 303 are shown by way of example only.

As with the previously-described embodiments, the vertical wall 308 (and particularly, a second major surface 317 thereof) can include a first mating surface 322 of a mechanical fastener 325, and the flap 306 (and, particularly, a first major surface 320 thereof) can include a second mating surface 326 of the mechanical fastener 325 configured to engage the first mating surface 322. In addition, by way of example, at least a portion of a first major surface 310 of the patch 302 can include the first mating surface 322.

By way of example only, the first mating surface 322 is schematically illustrated in FIG. 7 as including hooks or pegs, and the second mating surface 326 is schematically illustrated as including piles or loops. However, it should be understood that any appropriate mechanical fastener engagement surface or mechanism can be employed, as long as the first mating surface 322 and the second mating surface 326 are complementary mating surfaces of a mechanical fastener that are configured to engage one another.

The rigid securement device 330 is more rigid than the patch 302 (and the flap 306) and can also be referred to as a bracket, a retainer, a retaining element, a clip, a clamp, or a combination thereof The rigid securement device 330 is coupled to the patch 302, and particularly, to the first major surface 310 thereof. In some embodiments, as shown in FIG. 7, the rigid securement device 330 can be positioned between the flap 306 (e.g., a free end 316 thereof, particularly when the free end 316 of the flap 306 is in the first position) and the vertical wall 308.

In some embodiments, as shown in FIG. 7, the rigid securement device 330 can provide the vertical wall 308 (and, optionally, other securement feature(s) of the rigid securement device 330 can be located between the flap 306 and the vertical wall 308). In such embodiments, the vertical wall 308 can be formed by (or provided by, e.g., integrally formed with) a portion (e.g., an outer wall) of the rigid securement device 330, such that the first mating surface 322 is located on or provided by the rigid securement device, and particularly on an opposite side of the rigid securement device 330 from the flap 306. The first mating surface 322 can be formed directly on the rigid securement device 330 (e.g., an outer wall thereof that forms the vertical wall 308), or the first mating surface 322 can be provided by a material that is coupled to the rigid securement device 330.

In other embodiments, the vertical wall 308 can be provided separately from the rigid securement device 330 and can be coupled to the rigid securement device 330, e.g., to an outer vertical wall of the rigid securement device 330. For example, in some embodiments, a first major surface of the vertical wall (see, e.g., first major surface 113 of FIGS. 1-3) can be coupled to a portion (e.g., an outer wall) of the rigid securement device 330. Still, in other embodiments, the rigid securement device 330 (e.g., an outer wall thereof) can be positioned adjacent the vertical wall 308 (e.g., the first major surface thereof), and need not be coupled to the vertical wall 308.

In some embodiments, the rigid securement device 330 can be coupled to the patch 302 adjacent a fixed end of the flap 306, and in some embodiments, the rigid securement device 330 can be positioned atop at least a portion of a fixed end of the flap 306.

The rigid securement device 330 can include a variety of securement features specific to a medical article of interest. By way of example only, the rigid securement device 330 is shown as including a base 332 atop which a medical article can be positioned, and a clip 334. The clip 334 can include an arm 338 that is spaced a distance from the base 332 (and the patch 302) and an aperture or channel 336. The clip 334 can function as a longitudinal stop inhibiting longitudinal movement of a medical article. In addition, the clip 334 illustrated in FIG. 7 is configured and positioned on the patch 302 to further inhibit vertical movement and lateral movement in at least one lateral direction. In some embodiments, at least a portion of the medical article can be positioned in or through the aperture 336, e.g., such that the at least a portion of the clip 334 abuts an irregular feature of the medical article. By way of example, the arm 338 can be fixed relative to the base 332, such that the rigid securement device 330 includes no moving parts.

Each embodiment shown in the figures is illustrated as a separate embodiment for clarity in illustrating a variety of features of the medical article securement systems of the present disclosure. However, it should be understood that any combination of elements and features of any of the embodiments illustrated in the figures and described herein can be employed in the medical article securement systems of the present disclosure.

The following embodiments are intended to be illustrative of the present disclosure and not limiting.

### Embodiments

Embodiment 1 is a medical article securement system for securing a medical article, the system comprising:
a patch comprising
   a first major surface, and
   a second major surface, opposite the first major surface, comprising a skin-contact adhesive;
a vertical wall, the vertical wall comprising a first major surface positioned to face the medical article and a second major surface opposite the first major surface, the second major surface comprising a first mating surface of a mechanical fastener; and
a flap comprising
   a fixed end coupled to the patch, and
   a free end that is movable with respect to the patch between a first position in which the free end is not positioned in overlapping relationship with the vertical wall and a second position in which the free end is positioned in overlapping relationship with the vertical wall to couple at least a portion of the medical article to the patch, at least the free end comprising:
      a first major surface positioned to face the medical article, at least a portion of the first major surface comprising a second mating surface of a mechanical fastener configured to engage the first mating surface of the vertical wall, and
      a second major surface, opposite the first surface, positioned to face away from the medical article.

Embodiment 2 is the system of embodiment 1, wherein at least the free end of the flap is positioned to extend from the first major surface of the vertical wall.

Embodiment 3 is the system of embodiment 1 or 2, wherein the vertical wall is a first vertical wall, and further comprising a second vertical wall oriented substantially parallel with respect to first vertical wall.

Embodiment 4 is the system of any of embodiments 1-3, wherein the vertical wall is a first vertical wall, and further comprising a second vertical wall, wherein the first vertical wall and the second vertical wall are positioned to receive at least a portion of the medical article therebetween.

Embodiment 5 is the system of any of embodiments 1-4, further comprising a rigid securement device coupled to the patch that is more rigid than the flap.

Embodiment 6 is the system of embodiment 5, wherein the rigid securement device is positioned atop at least a portion of the fixed end of the flap.

Embodiment 7 is the system of embodiment 5 or 6, wherein the rigid securement device is positioned between the free end of the flap and the vertical wall when the free end of the flap is in the first position.

Embodiment 8 is the system of any of embodiments 5-7, wherein the vertical wall and the rigid securement device are coupled together.

Embodiment 9 is the system of any of embodiments 5-8, wherein the rigid securement device is positioned adjacent the first major surface of the vertical wall.

Embodiment 10 is the system of any of embodiments 5-9, wherein the first major surface of the vertical wall is coupled to the rigid securement device.

Embodiment 11 is the system of any of embodiments 5-7, wherein the rigid securement device includes the vertical wall.

Embodiment 12 is the system of any of embodiments 1-11, wherein the system is free of a rigid component that is more rigid than the flap.

Embodiment 13 is the system of any of embodiments 1-12, wherein at least a portion of the first major surface of the patch includes the first mating surface.

Embodiment 14 is the system of any of embodiments 1-13, wherein the vertical wall extends substantially orthogonally with respect to the patch.

Embodiment 15 is the system of any of embodiments 1-14, wherein the vertical wall and the flap are integrally formed.

Embodiment 16 is the system of embodiment 15, wherein the vertical wall is formed by a first end of the flap, and wherein the free end of the flap is formed by a second end of the flap that is opposite the first end.

Embodiment 17 is the system of any of embodiments 1-16, wherein at least the free end of the flap and the vertical wall are positioned to receive at least a portion of the medical article therebetween.

Embodiment 18 is the system of any of embodiments 1-17, wherein the vertical wall and the flap are formed of the same material.

Embodiment 19 is the system of any of embodiments 1-18, wherein the vertical wall is one of a plurality of vertical walls, and wherein the plurality of vertical walls are positioned to receive at least a portion of the medical article between two adjacent vertical walls.

Embodiment 20 is the system of any of embodiments 1-19, wherein the medical article includes a multi-lumen joint, and wherein the vertical wall is positioned to provide a longitudinal stop for the multi-lumen joint.

Embodiment 21 is the system of any of embodiments 1-20, wherein the medical article includes a multi-lumen joint, at least two proximal tubings connected to the multi-lumen joint and at least one distal tubing connected to the multi-lumen joint, and wherein the vertical wall is dimensioned to be received between two proximal tubings.

Embodiment 22 is the system of any of embodiments 1-21, further comprising a longitudinal axis configured to be substantially aligned with a longitudinal axis of the medical article;
wherein the vertical wall has a length that is greater than its width and its thickness, and wherein the length of the vertical wall is oriented substantially parallel with respect to the longitudinal axis; wherein the flap has a length that is greater than its width and its thickness, and wherein the length of the flap extends at least partially laterally with respect to the longitudinal axis.

Embodiment 23 is the system of any of embodiments 1-22, wherein the medical article includes an irregular feature, and wherein the flap is configured to be coupled to or adjacent the irregular feature.

Embodiment 24 is the system of any of embodiments 1-23, wherein the medical article includes an irregular feature, and wherein the vertical wall is configured to be positioned adjacent a portion of the medical article comprising the irregular feature.

Embodiment 25 is the system of any of embodiments 1-24, wherein the medical article includes a medical tubing.

Embodiment 26 is the system of embodiment 25, wherein the flap and the vertical wall are configured to be positioned adjacent the medical tubing.

Embodiment 27 is the system of embodiment 25 or 26, wherein the flap is configured to be wrapped about at least a portion of the circumference of the medical tubing.

Embodiment 28 is a medical article securement system for securing a medical article, the system comprising:
a longitudinal axis configured to be substantially aligned with a longitudinal axis of the medical article;
a patch comprising
   a first major surface configured to receive the medical article, and
   a second major surface, opposite the first major surface, comprising a skin-contact adhesive;
a vertical wall, the vertical wall comprising a first major surface positioned to face the medical article and a second major surface opposite the first major surface, the second major surface comprising a first mating surface of a mechanical fastener; and
a lateral flap comprising
   a fixed end coupled to the patch, and
   a free end that is movable with respect to the patch between a first position in which the free end is not positioned in overlapping relationship with the longitudinal axis and the vertical wall and a second position in which the free end is positioned in overlapping relationship with the longitudinal axis and the vertical wall to couple at least a portion of the medical article to the patch, at least the free end extending laterally from the first major surface of the vertical wall, at least the free end comprising:
      a first major surface positioned to face the medical article, at least a portion of the first major surface comprising a second mating surface of a mechanical fastener configured to engage the first mating surface of the vertical wall, and
      a second major surface, opposite the first surface, positioned to face away from the medical article.

It is to be understood that the invention is not limited in its application to the details of construction and the arrangement of components set forth in the above description or illustrated in the accompanying drawings. The invention is capable of other embodiments and of being practiced or of being carried out in various ways. Also, it is to be understood that the phraseology and terminology used herein is for the purpose of description and should not be regarded as limiting. It is to be further understood that other embodiments may be utilized, and structural or logical changes may be made without departing from the scope of the present disclosure.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1. A medical article securement system for securing a medical article, the system comprising:
a patch comprising
a first major surface, and
a second major surface, opposite the first major surface, comprising a skin-contact adhesive;
a vertical wall, the vertical wall comprising a first major surface positioned to face the medical article and a second major surface opposite the first major surface, the second major surface comprising a first mating surface of a mechanical fastener; and
a flap comprising
a fixed end coupled to the patch, and
a free end that is movable with respect to the patch between a first position in which the free end is not positioned in overlapping relationship with the vertical wall and a second position in which the free end is positioned in overlapping relationship with the vertical wall to couple at least a portion of the medical article to the patch, at least the free end comprising:
a first major surface positioned to face the medical article, at least a portion of the first major surface comprising a second mating surface of a mechanical fastener configured to engage the first mating surface of the vertical wall, and
a second major surface, opposite the first surface, positioned to face away from the medical article.

2. The system of claim 1, wherein at least the free end of the flap is positioned to extend from the first major surface of the vertical wall.

3. The system of claim 1, wherein the vertical wall is a first vertical wall, and further comprising a second vertical wall, wherein the first vertical wall and the second vertical wall are positioned to receive at least a portion of the medical article therebetween.

4. The system of claim 1, further comprising a rigid securement device coupled to the patch that is more rigid than the flap.

5. The system of claim 4, wherein the rigid securement device is positioned atop at least a portion of the fixed end of the flap.

6. The system of claim 4, wherein the rigid securement device is positioned between the free end of the flap and the vertical wall when the free end of the flap is in the first position.

7. The system of claim 4, wherein the vertical wall and the rigid securement device are coupled together.

8. The system of claim 4, wherein the rigid securement device is positioned adjacent the first major surface of the vertical wall.

9. The system of claim 4, wherein the rigid securement device includes the vertical wall.

10. The system of claim 1, wherein the system is free of a rigid component that is more rigid than the flap.

11. The system of claim 1, wherein at least a portion of the first major surface of the patch includes the first mating surface.

12. The system of claim 1, wherein the vertical wall extends substantially orthogonally with respect to the patch.

13. The system of claim 1, wherein the vertical wall and the flap are integrally formed.

14. The system of claim 13, wherein the vertical wall is formed by a first end of the flap, and wherein the free end of the flap is formed by a second end of the flap that is opposite the first end.

15. The system of claim 1, wherein the medical article includes a multi-lumen joint, and wherein the vertical wall is positioned to provide a longitudinal stop for the multi-lumen joint.

16. The system of claim 1, wherein the medical article includes a multi-lumen joint, at least two proximal tubings connected to the multi-lumen joint and at least one distal tubing connected to the multi-lumen joint, and wherein the vertical wall is dimensioned to be received between two proximal tubings.

17. The system of claim 1, further comprising a longitudinal axis configured to be substantially aligned with a longitudinal axis of the medical article;
wherein the vertical wall has a length that is greater than its width and its thickness, and wherein the length of the vertical wall is oriented substantially parallel with respect to the longitudinal axis; wherein the flap has a length that is greater than its width and its thickness, and wherein the length of the flap extends at least partially laterally with respect to the longitudinal axis.

18. The system of claim 2, wherein the vertical wall is a first vertical wall, and further comprising a second vertical wall, wherein the first vertical wall and the second vertical wall are positioned to receive at least a portion of the medical article therebetween.

19. The system of claim 2, further comprising a rigid securement device coupled to the patch that is more rigid than the flap.

20. The system of claim 3, further comprising a rigid securement device coupled to the patch that is more rigid than the flap.
